(19) United States
(12) Patent Application Publication
(10) Pub. No.: US 2026/0248578 A1
(43) Pub. Date: Aug. 27, 2026

(54) SYSTEMS AND METHODS FOR DETERMINING A FORCE APPLIED TO AN ANATOMICAL OBJECT WITHIN A SUBJECT BASED ON A DEFORMABLE THREE-DIMENSIONAL MODEL

(71) Applicant: Intuitive Surgical Operations, Inc., Sunnyvale, CA (US)

(72) Inventor: Kenneth K. Lee, Oakton, VA (US)

(21) Appl. No.: 19/106,735

(22) PCT Filed: Sep. 21, 2023
(86) PCT No.: PCT/US2023/033379

§ 371 Date: Feb. 26, 2025

Related U.S. Application Data
(60) Provisional application No. 63/409,981, filed on Sep. 26, 2022.

(51) Int. Cl. A61B 34/00; A61B 34/10; A61B 34/37; A61B 90/00

(52) U.S. Cl. CPC A61B 34/76; A61B 34/10; A61B 34/25; A61B 34/37; A61B 90/361; A61B 2034/104; A61B 2034/105; A61B 2090/062; A61B 2090/371

(57) ABSTRACT

An illustrative force determination system may be configured to detect an amount of deformation of a deformable 3D model of a scene that occurs when an anatomical object located in the scene is deformed and determine, based on the amount of deformation of the deformable 3D model, a force value representative of a force that caused the deformation of the anatomical object. The force determination system may further be configured to provide haptic feedback to a user based on the force value.

### RELATED APPLICATIONS

The present application claims priority to U.S. Provisional Patent Application No. 63/409,981, filed Sep. 26, 2022, the contents of which is hereby incorporated by reference in its entirety.

### BACKGROUND INFORMATION

In some scenarios, a computer-assisted medical system may be used to perform a medical procedure. As an example, one or more instruments may be at least partially inserted into a subject such that a surgeon may use a computer-assisted medical system to manipulate the one or more instruments within the subject.

Unfortunately, the surgeon may not receive feedback (e.g., haptic feedback) from the one or more instruments during the medical procedure, which may render it difficult for the surgeon to determine an amount of force that is being applied to internal anatomy of the subject by the one or more instruments. Such a lack of feedback from the one or more instruments may cause an undesired amount of force to be applied by the one or more instruments within the subject. Moreover, the surgeon may be unaware of instrument errors (e.g., stapler misfires) and/or a condition of the internal anatomy within the subject due to the lack of feedback from the one or more instruments.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its purpose is to present one or more aspects of the systems and methods described herein as a prelude to the detailed description that is presented below.

An illustrative system includes a memory storing instructions and one or more processors communicatively coupled to the memory. The one or more processors may be configured to execute the instructions to perform a process comprising: detecting an amount of deformation of a deformable three-dimensional (3D) model of a scene that occurs when an anatomical object located in the scene is deformed and determining, based on the amount of deformation of the deformable 3D model, a force value representative of a force that caused the deformation of the anatomical object.

An illustrative method includes detecting, by at least one computing device, an amount of deformation of a deformable three-dimensional (3D) model of a scene that occurs when an anatomical object located in the scene is deformed and determining, based on the amount of deformation of the deformable 3D model, a force value representative of a force that caused the deformation of the anatomical object.

An illustrative non-transitory computer-readable medium may store instructions that, when executed, direct a processor of a computing device to perform a process comprising: detecting an amount of deformation of a deformable three-dimensional (3D) model of a scene that occurs when an anatomical object located in the scene is deformed and determining, based on the amount of deformation of the deformable 3D model, a force value representative of a force that caused the deformation of the anatomical object.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative implementation including a force determination system.

FIG. 2 shows another illustrative implementation including a force determination system.

FIG. 3 shows an illustrative method of operating a force determination system.

FIG. 4 shows another illustrative method of operating a force determination system.

FIGS. 5A and 5B show illustrative implementations of generating a deformable 3D model using a force determination system.

FIGS. 6A and 6B show illustrative implementations of determining a force value using a force determination system.

FIG. 7 shows an illustrative implementation of a display that may be generated using a force determination system.

FIG. 8 shows an illustrative computer-assisted medical system that may incorporate a force determination system.

FIG. 9 shows an illustrative computing system according to principles described herein.

### DETAILED DESCRIPTION

An illustrative force determination system may be configured to determine an amount of force that causes deformation of an anatomical object within a scene (e.g., an area within a subject of a medical procedure) based on a deformable 3D model of the scene. For example, the force determination system may be configured to detect an amount of deformation of the deformable 3D model that occurs when an anatomical object located in the scene is deformed and determine, based on the amount of deformation of the deformable 3D model, a force value representative of a force that caused the deformation of the anatomical object.

In some implementations, the deformable 3D model may be generated in real-time during the medical procedure based on imagery (e.g., as captured by an imaging device) of the scene. The deformable 3D model may be deformed to depict movement of the anatomical object within the scene as the anatomical object deforms (e.g., due to a force applied by an instrument during a medical procedure). This may allow the force determination system to determine the force value visually based on the amount of deformation of the deformable 3D model. In some implementations, haptic feedback representative of the force may be provided to a user (e.g., a surgeon using the instrument to apply the force).

The principles described herein may result in improved force determinations compared to conventional techniques that are not based on a deformable 3D model, as well as provide other benefits as described herein. For example, the determination of a force value based on a deformable 3D model may allow the force value to be determined more accurately and/or efficiently. To illustrate, the determination of the force value based on a deformable 3D model may be used to provide real-time and realistic haptic feedback to the surgeon such that a desired amount of force may be applied by the one or more instruments within the subject. Moreover, the determination of the force value based on a deformable 3D model may allow the surgeon to be aware of instrument errors (e.g., stapler misfires) and/or a condition of the internal anatomy within the subject.

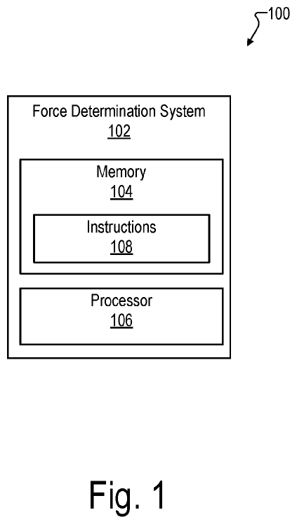

FIG. 1 shows an illustrative implementation 100 configured to determine a force value representative of a force that caused deformation of an anatomical object within a scene based on a deformable 3D model of the scene. As shown, implementation 100 includes a force determination system 102 configured to generate, based on imagery of a scene, a deformable 3D model of the scene, detect an amount of deformation of the deformable 3D model that occurs when an anatomical object located in the scene is deformed, and determine, based on the amount of deformation of the deformable 3D model, a force value representative of a force that caused the deformation of the anatomical object. Implementation 100 may include additional or alternative components as may serve a particular implementation. In some examples, implementation 100 or certain components of implementation 100 may be implemented by a computer-assisted medical system.

Force determination system 102 may be implemented by one or more computing devices and/or computer resources (e.g., processors, memory devices, storage devices, etc.) as may serve a particular implementation. As shown, force determination system 102 may include, without limitation, a memory 104 and a processor 106 selectively and communicatively coupled to one another. Memory 104 and processor 106 may each include or be implemented by computer hardware that is configured to store and/or process computer software. Various other components of computer hardware and/or software not explicitly shown in FIG. 1 may also be included within force determination system 102. In some examples, memory 104 and/or processor 106 may be distributed between multiple devices and/or multiple locations as may serve a particular implementation.

Memory 104 may store and/or otherwise maintain executable data used by processor 106 to perform any of the functionality described herein. For example, memory 104 may store instructions 108 that may be executed by processor 106. Memory 104 may be implemented by one or more memory or storage devices, including any memory or storage devices described herein, that are configured to store data in a transitory or non-transitory manner. Instructions 108 may be executed by processor 106 to cause force determination system 102 to perform any of the functionality described herein. Instructions 108 may be implemented by any suitable application, software, code, and/or other executable data instance. Additionally, memory 104 may also maintain any other data accessed, managed, used, and/or transmitted by processor 106 in a particular implementation.

Processor 106 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 106 (e.g., when processor 106 is directed to perform operations represented by instructions 108 stored in memory 104), force determination system 102 may perform various operations as described herein.

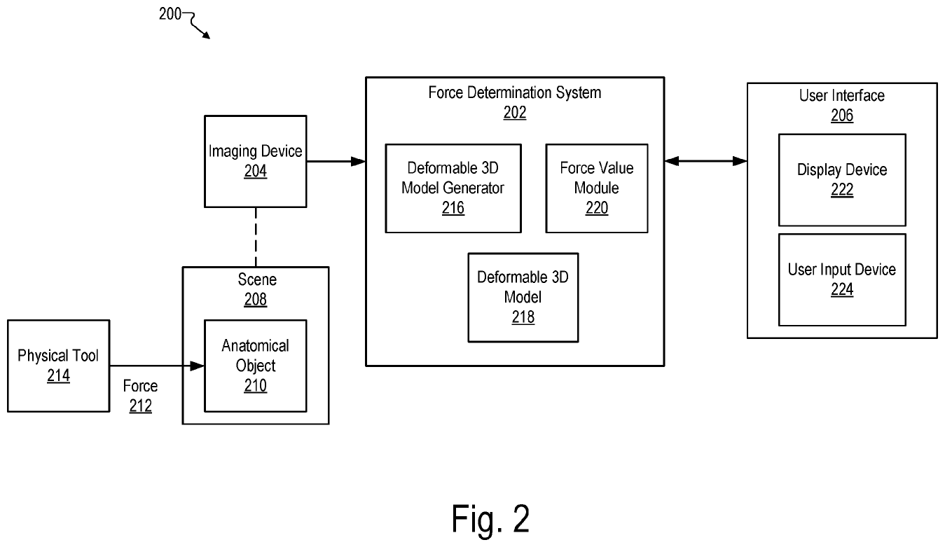

FIG. 2 shows another illustrative implementation 200 configured to determine a force value representative of a force that caused deformation of an anatomical object within a scene based on a deformable 3D model of the scene. As shown, implementation 200 includes a force determination system 202 communicatively coupled (e.g., wired and/or wirelessly) with an imaging device 204 and a user interface 206. Implementation 200 may include additional or alternative components as may serve a particular implementation. In some examples, implementation 200 or certain components of implementation 200 may be implemented by a computer-assisted medical system.

Imaging device 204 may be implemented by an endoscope or other suitable device configured to capture and output imagery (e.g., images, videos, a sequence of image frames, etc.) of a scene 208. In some implementations, imaging device 204 may include, but is not limited to, one or more of: video imaging devices, infrared imaging devices, visible light imaging devices, non-visible light imaging devices, intensity imaging devices (e.g., color, grayscale, black and white imaging devices), depth imaging devices (e.g., stereoscopic imaging devices, time-of-flight imaging devices, infrared imaging devices, red-green-blue (RGB) imaging devices, red-green-blue and depth (RGB-D) imaging devices, light detection and ranging (LIDAR) imaging devices, etc.).

In some implementations, the imagery may include image data (e.g., color, grayscale, saturation, intensity, brightness, depth, etc.) captured by imaging device 204. The image data may, in some instances, be associated with data points expressed in a common coordinate frame such as 3D voxels or two-dimensional (2D) pixels of images captured by imaging device 204. In some implementations, imaging device 204 may be moved relative to scene 208 to capture imagery of scene 208 at different viewpoints.

Scene 208 may include an environment (e.g., an area within a subject of a medical procedure) and/or one or more objects within an environment. For example, scene 208 may include an anatomical object 210. Anatomical object 210 may include an object associated with a subject (e.g., a body of a live animal, a human or animal cadaver, a portion of human or animal anatomy, tissue removed from human or animal anatomies, non-tissue work pieces, training models, etc.). In some implementations, anatomical object 210 may include tissue of a subject (e.g., an organ, soft tissue, connective tissue, etc.).

Imaging device 204 may capture a deformation of anatomical object 210 within scene 208. To illustrate, a force 212 may be applied to anatomical object 210 that may cause anatomical object 210 to deform. Force 212 may be applied to anatomical object 210 by one or more physical tools 214 (e.g., instruments, scalpels, scissors, forceps, clamps, etc.) and/or other objects. While the illustrated implementation shows physical tool 214 positioned outside of scene 208, physical tool 214 may additionally or alternatively be included within scene 208. Still other non-anatomical objects (e.g., staples, mesh, sponges, etc.) used for a medical procedure may be included within scene 208.

Force determination system 202 may implement or be similar to force determination system 102 and may be configured to receive imagery of scene 208 from imaging device 204. In some implementations, force determination system 202 may be configured to fuse imagery of scene 208 captured by imaging device 204 at different viewpoints of scene 208. In certain examples, the fusing may include merging aligned (or overlapping) voxels or pixels, such as by blending intensity and/or depth values for aligned voxels or pixels. The blending may include weighted blending in which the data points being blended are weighted based on one or more factors, such as which camera of a stereoscopic device has the best view of a data point (e.g., by more heavily weighting data captured by the camera with the best viewing angle). The fusing may additionally or alternatively include stitching non-overlapping voxels or pixels together, such as by stitching images together along non-overlapping boundaries of the images. Accordingly, the fusing of imagery at different viewpoints may allow the imagery of scene 208 to include an area that is larger than a single field of view of imaging device 204.

As shown, force determination system 202 includes a deformable 3D model generator 216 configured to generate a deformable 3D model 218 based on imagery of scene 208. For example, deformable 3D model generator 216 may be configured to generate a point cloud having a plurality of nodes representative of surface points on one or more objects (e.g., anatomical object 210) within scene 208 as depicted in imagery captured by imaging device 204. Deformable 3D model generator 216 may further be configured to generate vertices associated with 3D locations that correspond to 3D locations of the plurality of nodes from the imagery. In instances where the plurality of nodes is based on 2D imagery, deformable 3D model generator 216 may be configured to determine a depth associated with the plurality of nodes, such as by processing stereoscopic images captured by imaging device 204. Additionally or alternatively, a depth map of scene 208 may be generated using a depth sensor.

Deformable 3D model generator 216 may further be configured to deform deformable 3D model 218 over time with the movement of one or more objects within scene 208. For example, anatomical object 210 within scene 208 may be deformed during a medical procedure (e.g., due to force 212) such that deformable 3D model 218 may be deformed to correspond to the deformation of anatomical object 210. To illustrate, the 3D locations of the vertices of deformable 3D model 218 may track the 3D locations of the plurality of nodes associated with the vertices as the 3D locations of the plurality of nodes update in the imagery captured by imaging device 204 with the movement of the one or more objects within scene 208. This may allow deformable 3D model 218 to deform over time with the movement of the one or more objects within scene 208. In some implementations, deformable 3D model generator 216 may be configured to detect deformation of deformable 3D model 218, such as by comparing the 3D locations of the vertices of deformable 3D model 218 at two or more different points of time. Additionally or alternatively, a first 3D model, which may be deformable or nondeformable, may be generated at a first point of time and a second 3D model, which may be deformable or nondeformable, may be generated at a second point of time that is different than the first point of time such that the first and second 3D models may be compared with each other to detect deformation.

In some implementations, a simultaneous localization and mapping (SLAM) heuristic may be used by deformable 3D model generator 216 to construct and/or update a map of scene 208 while simultaneously keeping track of the location of objects within scene 208. For example, the SLAM heuristic may be configured to generate the point cloud having the plurality of nodes representative of surface points on one or more objects within scene 208 and derive and/or associate vertices of deformable 3D model 218 with 3D locations that correspond to 3D locations of the plurality of nodes as imaging device 204 views scene 208 in real-time. The SLAM heuristic may further be configured to derive and/or associate additional vertices of deformable 3D model 218 with 3D locations that correspond to 3D locations of additional nodes as imaging device 204 is moved relative to scene 208 to capture additional areas of scene 208, while also tracking the 3D locations of the previous vertices of deformable 3D model 218 with the 3D locations of the previous nodes associated as one or more objects within scene 208 move and/or deform. In some implementations, the SLAM heuristic may be configured to track a pose of imaging device 204 (e.g., using vision software) while imaging device 204 is moved relative to scene 208.

Still other suitable configurations may be used to generate and/or update deformable 3D model 218 with movement of one or more objects within scene 208. For example, deformable 3D model generator 216 may be configured to generate deformable 3D model 218 based on preoperative imagery of scene 208. Additionally or alternatively, the movement of one or more objects within scene 208 may be determined based on kinematic data representative of movement of the one or more objects over time. For example, the kinematic data may be generated by or associated with a computer-assisted medical system communicatively coupled with the one or more objects (e.g., physical tool 214).

Force determination system 202 further includes a force value module 220 configured to determine, based on an amount of deformation of deformable 3D model 218, a force value representative of a force (e.g., force 212) that caused deformation of anatomical object 210 in scene 208. The force value may be represented by any suitable value, such as a discrete value (e.g., an integer, a range, a level, a percentage, etc.) representative of the force applied to anatomical object 210.

To illustrate, force value module 220 may be configured to detect an amount of deformation of deformable 3D model 218 that occurs when anatomical object 210 located in scene 208 is deformed, such as by force 212 applied to anatomical object 210 by physical tool 214. For example, force value module 220 may be configured to track, based on the imagery of scene 208 captured by imaging device 204, movement of the plurality of nodes representative of surface points on anatomical object 210 over time while force 212 is applied to anatomical object 210. Force value module 220 may further be configured to update the 3D locations of vertices of deformable 3D model 218 that are associated with the 3D locations of the plurality of nodes and compare the updated 3D locations of the vertices with the previous 3D locations of the vertices to determine the amount of deformation of deformable 3D model 218. Based on the amount of deformation of deformable 3D model 218, force value module 220 may determine a force value representative of force 212 that caused the deformation of anatomical object 210. In some implementations, the force value may represent an amount of physical force applied to anatomical object 210. Additionally or alternatively, the force value may represent a relative force based on changes in the deformation of anatomical object 210. As an illustrative example, the force value may increase as the amount of deformation increases and/or the force value may decrease as the amount of deformation decreases.

User interface 206 may be configured to receive the force value from force determination system 202. User interface 206 of the illustrated implementation includes a display device 222 and a user input device 224. Display device 222 may be implemented by a monitor or other suitable device configured to display information to a user. For example, display device 222 may be configured to display the force value received from force determination system 202. In some implementations, display device 222 may further be configured to display imagery of scene 208 captured by imaging device 204 and/or deformable 3D model 218 generated by force determination system 202. User input device 224 may include any suitable device (e.g., a button, joystick, touchscreen, keyboard, handle, etc.) configured to receive a user input such as to manipulate physical tool 214.

In some implementations, force determination system 202 may be configured to determine multiple force values based on deformable 3D model 218. As an illustrative example, force determination system 202 may be configured to determine a force value based on multiple areas of deformation of one or more anatomical objects 210 within scene 208 that may be caused by one or more physical tools 214. Moreover, force determination system 202 may be configured to mark, track, and/or present the multiple force values. For example, force determination system 202 may be configured to mark (e.g., highlight) the objects, such as anatomical objects 210 and/or physical tools 214, within scene 208 that are involved in determining the force values. Force determination system 202 may further be configured to track and update the multiple force values as one or more physical tools 214 are moved relative to one or more anatomical objects 210. Force determination system 202 may further be configured to present the multiple force values to a user, such as on display device 222. In some implementations, the multiple force values may be labeled on display device 222 for reference.

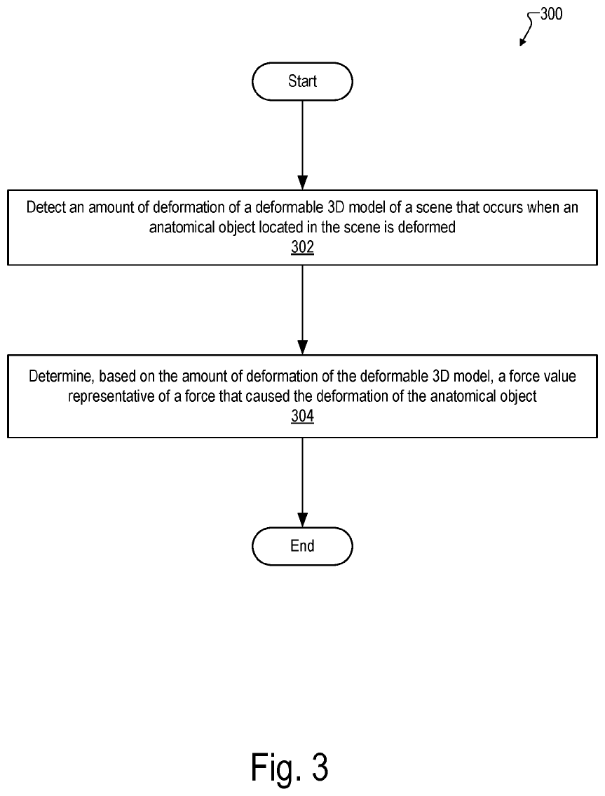

FIG. 3 shows an illustrative method 300 that may be performed by force determination system 202. While FIG. 3 illustrates exemplary operations according to one embodiment, other embodiments may omit, add to, reorder, and/or modify any of the operations shown in FIG. 3. Moreover, each of the operations depicted in FIG. 3 may be performed in any of the ways described herein.

As shown, force determination system 202 may, at operation 302, detect an amount of deformation of deformable 3D model 218 of scene 208 that occurs when anatomical object 210 located in scene 208 is deformed. In some implementations, force determination system 202 may generate, based on imagery of scene 208 captured by imaging device 204, a point cloud having a plurality of nodes representative of surface points on anatomical object 210 within scene 208 and derive vertices associated with 3D locations that correspond to 3D locations of the plurality of nodes (e.g., using a SLAM heuristic). The 3D locations of the vertices of deformable 3D model 218 may update with the corresponding 3D locations of the plurality of nodes as the 3D locations of the plurality of nodes move with the movement of anatomical object 210 within scene 208. This may allow deformable 3D model 218 to deform over time with the deformation of anatomical object 210 within scene 208.

Force determination system 202 may be configured to track, based on the imagery of scene 208 captured by imaging device 204, movement of the 3D locations of the plurality of nodes over time (e.g., while force 212 is applied to anatomical object 210). Force determination system 202 may further be configured to update the 3D locations of vertices of deformable 3D model 218 that are associated with the 3D locations of the plurality of nodes and compare the updated 3D locations of the vertices with the previous 3D locations of the vertices to determine the amount of deformation of deformable 3D model 218. In some implementations, the determining the amount of deformation may further include determining displacement values of the vertices (e.g., a change in location of the vertices in a direction, angle, velocity, acceleration, etc.) caused by force 212 being applied by physical tool 214 to anatomical object 210.

Force determination system 202 may further, at operation 304, determine, based on the amount of deformation of deformable 3D model 218, a force value representative of force 212 that caused the deformation of anatomical object 210. For example, the amount of deformation of deformable 3D model 218 may indicate an amount of force being applied to anatomical object 210. As an illustrative example, the force value may increase as the amount of deformation increases and/or the force value may decrease as the amount of deformation decreases. Still other suitable methods may be used for determining the force value.

To illustrate, the determining the force value may further be based on one or more material property values of deformable 3D model 218 that may be representative of one or more material properties of anatomical object 210. For example, material properties (e.g., a mass, stiffness, stress, strain, strength, hardness, elasticity, Young's modulus, Poisson Ratio, etc.) of anatomical object 210 may contribute to how anatomical object 210 deforms in response to force 212 (e.g., based on Hooke's Law, F=kx, where F is a force, k is a constant based on material properties, and x is an amount of deformation). In some implementations, deformable 3D model 218 may include baseline material property values that may be used for any anatomical object 210, predetermined material property values that may be used for an anatomical object 210 with known material properties, and/or relative material property values that may vary between different areas of anatomical object and/or different anatomical objects 210. For example, material property values may vary between a more rigid anatomical object 210 (e.g., that may show a lower amount of deformation) and a more flexible anatomical object 210 (e.g., that may show a higher amount of deformation). In some implementations, the material property values of deformable 3D model 218 may be adjusted (e.g., from an initial baseline material property value to a predetermined or relative material property value).

The material property values may be associated with vertices of deformable 3D model 218. For example, the material property values at the vertices of deformable 3D model 218 may be constant throughout the vertices and/or the material property values may vary between the vertices of deformable 3D model 218. For example, some anatomical objects 210 may have inhomogeneous material properties such that the material properties may vary throughout the anatomical objects 210 at various vertices. In instances where anatomical object 210 includes inhomogeneous material properties, one or more material property values may be associated with a region of anatomical object 210. To illustrate, a first material property value may be representative of a first material property associated with a first region of anatomical object 210 and a second material property value may be representative of a second material property associated with a second region of anatomical object 210 and/or a first material property value may be representative of a material property associated with a first region of anatomical object 210 and a second material property value may be representative of the same material property associated with a second region of anatomical object 210. Additionally, force determination system 202 may be configured to determine which region of anatomical object 210 is deformed and/or in contact with physical tool 214 such that force determination system 202 may use the one or more material property values associated with that region of anatomical object 210 in determining the force value.

In some implementations, the determining the force value may further include implementing and applying artificial intelligence algorithms, such as machine learning algorithms, to identify a type of anatomical object 210 (e.g., an organ (e.g., a kidney, intestines, etc.), tissue (e.g., connective tissue, muscle, nervous tissue, etc.), etc.). In some implementations, the identified type of anatomical object 210 may be used to associate material property values with deformable 3D model 218 (e.g., based on known material properties of the identified type of anatomical object 210). Any suitable form of artificial intelligence and/or machine learning may be used, including, for example, deep learning, neural networks, etc. For example, a machine learning algorithm may be generated through machine learning procedures and applied to identification operations. The machine learning algorithm may operate as an identification function that is applied to individual and/or fused imagery to classify anatomical object 210 in the imagery.

Still other suitable methods may be used for identifying a type of anatomical object 210 in addition to or instead of machine learning algorithms. For example, force determination system 202 may be configured to identify the type of anatomical object 210 within scene 208 by implementing and applying object recognition algorithms. For example, an object recognition algorithm may be used to identify objects (e.g., anatomical object 210) of predetermined types within the image data received from imaging device 204, such as by comparing the image data received from imaging device 204 to model object data of predetermined types of objects. Such model object data may be stored within a model database that may be communicatively coupled with force determination system 202.

In some implementations, the determining the force value may further be based on a position of physical tool 214 applying force 212 to cause the deformation of anatomical object 210. For example, the detecting the position of physical tool 214 may be based on kinematic data representative of movement of physical tool 214 over time. Such kinematic data may be generated by or associated with a computer-assisted medical system communicatively coupled with physical tool 214. Accordingly, the known position of physical tool 214 relative to anatomical object 210 may allow force determination system 202 to determine when physical tool 214 is pressing on anatomical object 210.

Additionally, the determining the force value may be based on detecting the amount of deformation due to the position of physical tool 214. For example, the known location of physical tool 214 may be used to determine the amount of deformation that occurs at or near the known position of physical tool 214, which may allow force determination system 202 to determine the amount of deformation induced by physical tool 214 and estimate the associated force 212. In some implementations, force determination system 202 may only compute the amount of deformation induced by physical tool 214 at the known position of physical tool 214 (e.g., to reduce processing burden). Additionally or alternatively, the determining the force value may further be based on one or more physical properties of physical tool 214 (e.g., a type, a material, a size, etc.).

In some implementations, the force value may be based on detecting the amount of deformation due to motion of physical tool 214. For example, force determination system 202 may be configured to determine a movement of physical tool 214 applying the force to cause the deformation of anatomical object 210 such that the determining the force value may further be based on the movement of physical tool 214. Additionally, the detecting the amount of deformation may include determining an amount of tool-induced deformation (e.g., deformation of anatomical object 210 induced by physical tool 214) based on the amount of deformation of deformable 3D model 218 and the movement of physical tool 214 such that the determining the force value may be based on the amount of the tool-induced deformation. The determining the movement of physical tool 214 may be based on kinematic data representative of movement of physical tool 214 over time. The kinematic data may be generated by a computer-assisted medical system communicatively coupled with physical tool 214.

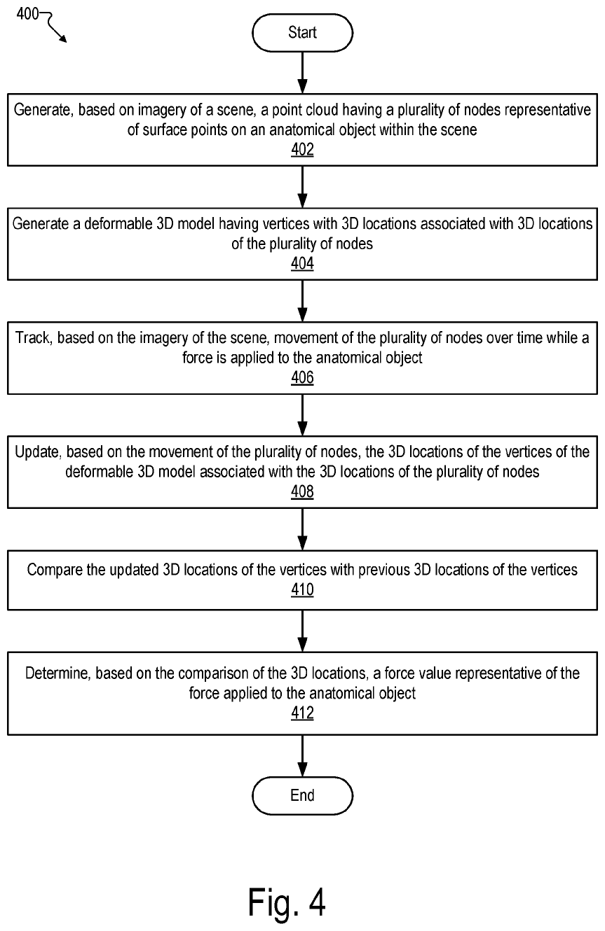

FIG. 4 shows another illustrative method 400 that may be performed by force determination system 202. While FIG. 4 illustrates exemplary operations according to one embodiment, other embodiments may omit, add to, reorder, and/or modify any of the operations shown in FIG. 4. Moreover, each of the operations depicted in FIG. 4 may be performed in any of the ways described herein.

As shown, force determination system 202 may, at operation 402, be configured to generate, based on imagery of scene 208 (e.g., as captured by imaging device 204), a point cloud having a plurality of nodes representative of surface points on anatomical object 210 within scene 208. Force determination system 202 may further, at operation 404, be configured to generate deformable 3D model 218 having vertices with 3D locations associated with 3D locations of the plurality of nodes.

Force determination system 202 may further, at operation 406, track, based on the imagery of scene 208, movement of the plurality of nodes over time while force 212 is applied to anatomical object 210. Force determination system 202 may further, at operation 408, update, based on the movement of the plurality of nodes, the 3D locations of the vertices of deformable 3D model associated with the 3D locations of the plurality of nodes. Force determination system 202 may further, at operation 410, compare the updated 3D locations of the vertices with previous 3D locations of the vertices (e.g., to determine the amount of deformation of deformable 3D model 218).

Force determination system 202 may further, at operation 412, determine, based on the comparison of the 3D locations, a force value representative of force 212 applied to anatomical object 210. In some implementations, force determination system 202 may dynamically update the force value with movement of physical tool 214 and/or anatomical object 210. For example, the 3D locations of the vertices of deformable 3D model 218 associated with the plurality of nodes may move as anatomical object 210 is deformed. These changes in the 3D locations of the vertices may affect the amount of deformation of deformable 3D model 218 such that the force value may be dynamically updated. In some scenarios, force determination system 202 may only determine and/or update the force value if a change in the amount of deformation of deformable 3D model 218 has been detected (e.g., to reduce processing burden).

In some implementations, the force value may be dynamically updated based on the 3D locations of the vertices corresponding to each sequential image frame of the imagery captured by imaging device 204. Additionally or alternatively, the force value may be dynamically updated based on the 3D locations of the vertices corresponding to a plurality of image frames over time. For example, the force value may represent a combination (e.g., an average, a mean, a median, etc.) of force measurements over the plurality of image frames.

In some implementations, method 400 may further include performing, by force determination system 202, an operation based on the force value. For example, force determination system 202 may be configured to provide haptic feedback to a user based on the force value. Such haptic feedback may include audio feedback, visual feedback, and/or tactile feedback. Audio feedback may include an audio output (e.g., a noise, a beep, etc.) that may sound based on the force value.

Visual feedback may include displaying the force value one or more display devices (e.g., display device 222). For example, the force value may be displayed as a readout on the one or more display devices. In some implementations, force determination system 202 may further be configured to instruct one or more display devices (e.g., display device 222) to display the imagery depicting scene 208 and/or deformable 3D model 218 such that the force value may be labeled on the imagery depicting scene 208 and/or deformable 3D model 218. Additionally or alternatively, the visual feedback may include shading and/or color coding (e.g., red, yellow, green, etc.) representative of the force value on the imagery depicting scene 208 and/or deformable 3D model 218. For example, areas of deformable 3D model 218 having a larger force value may be darker than areas of deformable 3D model 218 having a lower force value.

Tactile feedback may include adjusting a resistance and/or degree of freedom of a user input device 224 that may be used to manipulate physical tool 214. As an illustrative example, a degree of freedom of user input device 224 may be constrained in a direction of anatomical object 504 based on the force value.

In some implementations, the degree of the haptic feedback may be based on the force value such that the degree of the haptic feedback increases or decreases as the force value respectively increases or decreases. As an illustrative example, a constraint on the degree of freedom of user input device 224 may increase or decrease as the force value respectively increases or decreases.

Additionally or alternatively, the force value may be used to identify errors with respect to physical tool 214. To illustrate, monitoring the amount of deformation of deformable 3D model 218 may indicate when a stapler misfire and/or misalignment has occurred on anatomical object 210 (e.g., if the amount of deformation of anatomical object 210 depicted by deformable 3D model 218 differs from a typical amount of deformation caused by the insertion of a staple). If a misfire and/or misalignment has occurred, force determination system 202 may provide a notification or alert to a user and/or inhibit the operation of physical tool 214 to fire another staple.

As another example, the force value may be used to determine a degree of health of anatomical object 210. For example, tissue of anatomical object 210 may calcify or harden when a degree of health decreases. Accordingly, an increase in the force value may, in some instances, indicate a decrease in the degree of health of anatomical object 210. Additionally or alternatively, a higher force value may indicate a feature of anatomical object. To illustrate, a higher force value may indicate the location of a duct within anatomical object 210 such that it may not be desirable for the user to cut through the duct. In some implementations, force determination system 202 may provide a notification when the force value exceeds a threshold.

In some implementations, the operation may include providing a notification when the force value exceeds or falls below a threshold. For example, the threshold may be associated with a successful stapler fire such that the notification may indicate a stapler misfire. Additionally or alternatively, the threshold may be associated with a healthy anatomical object such that the notification may indicate an unhealthy anatomical object 210.

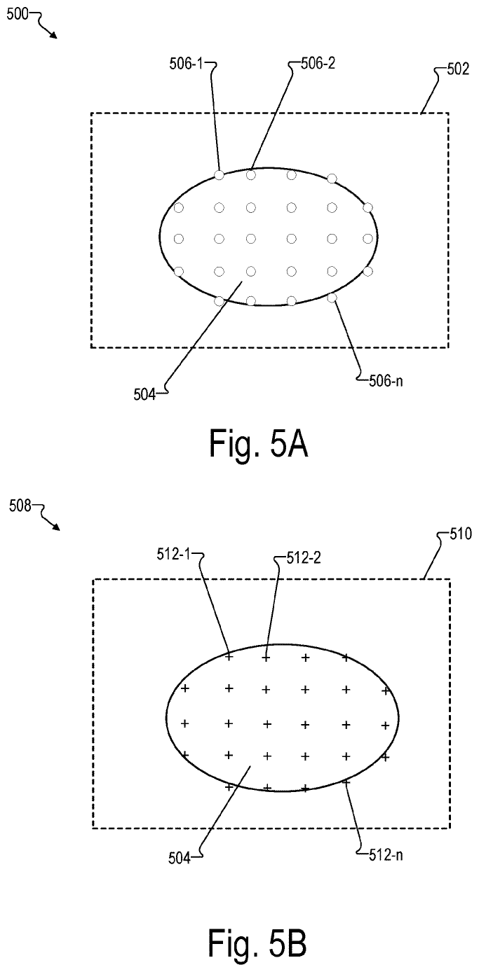

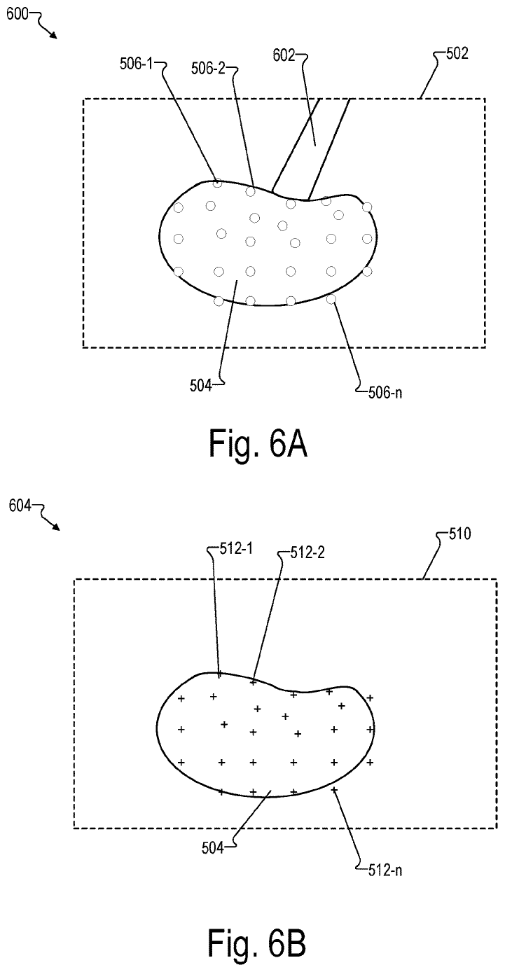

FIGS. 5A-6B show an illustrative example of determining a force value that may be performed by force determination system 202. For example, FIG. 5A shows an implementation 500 of imagery 502 of a scene (e.g., scene 208) that may be captured by imaging device 204. As shown, imagery 502 includes an anatomical object 504. In the illustrated implementation 500, a point cloud has been generated by force determination system 202 that includes a plurality of nodes 506 (e.g., nodes 506-1 to 506-n). Nodes 506 may be representative of surface points on anatomical object 504 within the scene captured by imagery 502. While the illustrated implementation shows a single anatomical object 504 located in the scene, other implementations may include additional anatomical objects 504 located in the scene.

FIG. 5B shows an illustrative implementation 508 of a deformable 3D model 510 that may be generated by force determination system 202 based on imagery 502. Deformable 3D model 510 may implement or be similar to deformable 3D model 218. As shown, force determination system 202 may generate deformable 3D model 510 by deriving vertices 512 (e.g., vertices 512-1 to 512-n) with 3D locations associated with the 3D locations of the plurality of nodes 506. Accordingly, deformable 3D model 510 may depict anatomical object 504 within the scene as captured by imagery 502. In some implementations, deformable 3D model 510 may be generated using a SLAM algorithm that may derive vertices 512 and track the location of vertices 512 as imaging device 204 captures imagery 502.

In some implementations, the deformation of anatomical object 504 within the scene depicted by imagery 502 may cause deformable 3D model 510 to deform. To illustrate, FIG. 6A shows another implementation 600 of imagery 502 that may be captured by imaging device 204 of anatomical object 504. As shown, anatomical object 504 has moved to a deformed state. The deformation of anatomical object 504 may be caused by a force applied by physical tool 602 to anatomical object 504. Physical tool 602 may implement or be similar to physical tool 214. The deformation of anatomical object 504 may cause the 3D locations of one or more of nodes 506 to move with the deformation of anatomical object 504.

FIG. 6B shows another implementation 604 of deformable 3D model 510 in a deformed state that may correspond to the deformed state of anatomical object 504. As shown, the 3D locations of vertices 512 of deformable 3D model 510 have been updated with the 3D locations of nodes 506 associated with vertices 512. The updated 3D locations of vertices 512 may allow deformable 3D model 510 to deform with the deformation of anatomical object 504.

Force determination system 202 may determine the force value based on the amount of deformation of deformable 3D model 510. For example, force determination system 202 may compare the updated 3D locations of vertices 512 (e.g., the 3D locations of vertices 512 in FIG. 6B) with previous 3D locations of vertices 512 (e.g., the 3D locations of vertices 512 in FIG. 5B) to determine the force value. In some implementations, the force value may further be based on other factors (e.g., the position of physical tool 602 relative to anatomical object 504, material property values representative of material properties of anatomical object 504, a type of anatomical object 504, an amount of deformation at or near physical tool 602, etc.) in addition to the amount of deformation of deformable 3D model 510.

In some implementations, the force value may be computed at each vertex 512 and/or a group of vertices 512 to provide force values at different areas of deformable 3D model 510. Alternatively, the force value may be computed as a general force applied to anatomical object 504 (e.g., a maximum force, an average force, a median force, a mean force, a minimum force, etc.). In some implementations, the force value may be recomputed with a change in the amount of deformation of deformable 3D model 510 and/or movement of physical tool 602 relative to anatomical object 504.

In the illustrated implementation, physical tool 602 is not included in deformable 3D model 510. For example, physical tool 602 may be removed in the generation of deformable 3D model 510 because the position of physical tool 602 may be known such that any nodes 506 associated with physical tool 602 may be removed and/or vertices 512 may not be associated with those nodes 506. Alternatively, physical tool 602 may be included in deformable 3D model 510.

In some instances, deformable 3D model 510 may be incomplete (e.g., in areas not captured by imaging device 204) such that there may be missing vertices (e.g., vertices 512). In these instances, force determination system 202 may be configured to perform a dynamic interpolation to estimate a 3D location for the missing vertices. For example, force determination system 202 may interpolate the 3D locations of the missing vertices based on the 3D locations of nearby vertices. Moreover, force determination system 202 may update the 3D locations of the missing vertices based on the movement of the nearby vertices with movement of the anatomical object within the scene. Force determination system 202 may be configured to perform the dynamic interpolation when the deformation of the anatomical object occurs in the incomplete area of deformable 3D model 510 (e.g., to reduce processing burden).

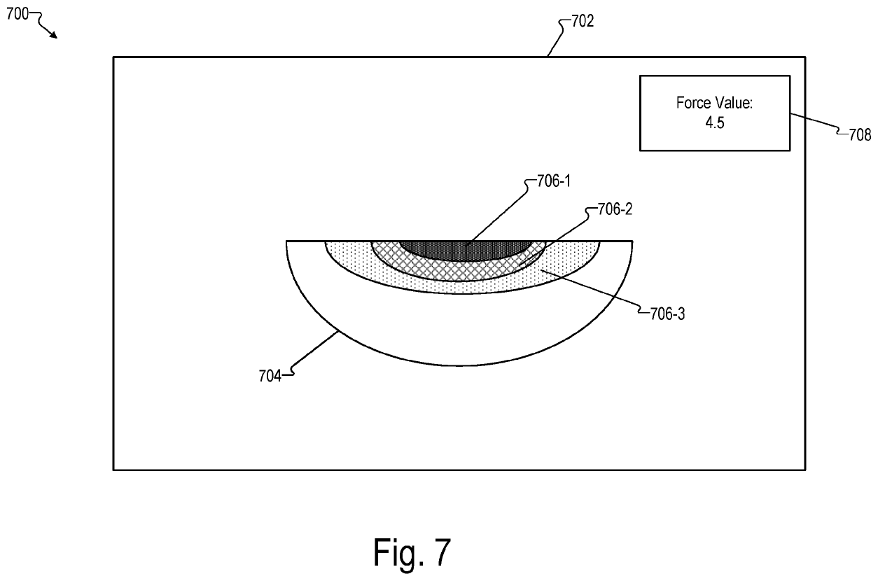

FIG. 7 shows an illustrative implementation 700 of a display 702 that may be displayed on display device 222. As shown, display 702 includes a display of a deformable 3D model 704 that may be generated by force determination system 202. Deformable 3D model 704 may implement or be similar to deformable 3D model 218 and/or deformable 3D model 510.

Display 702 may further depict levels of shading 706 (e.g., shading 706-1 to 706-3) that may represent a degree of the force that caused deformation of deformable 3D model 704. For example, a first level of shading 706-1 may depict a darker shade that may represent a higher degree of force than a second level of shading 706-2 and/or a third level of shading 706-3. Display 702 may further include a reference label 708 that may display the force value. In some implementations, display 702 may be updated in real-time such that shading 706 and/or label 708 depicting the force value may be updated as the deformation of deformable 3D model 704 changes.

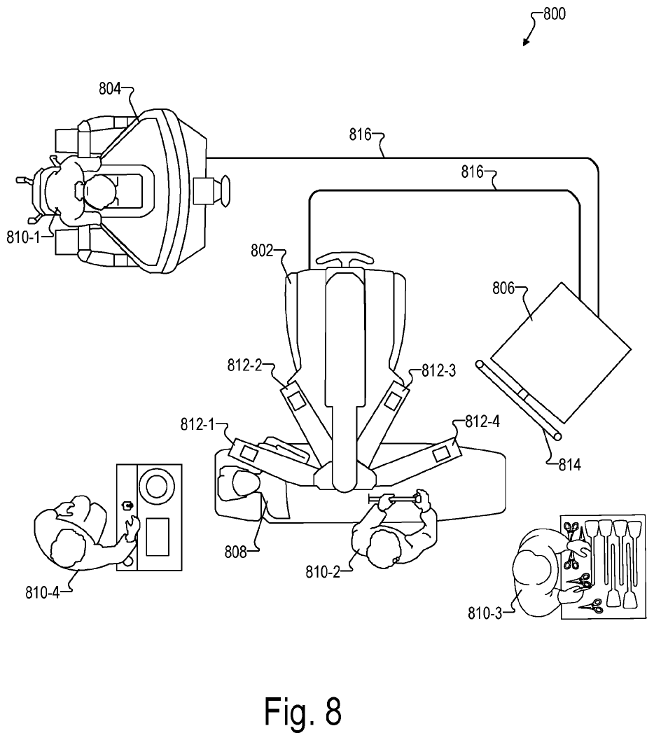

As has been described, force determination system 202, imaging device 204, user interface 206, and/or physical tool 214 may be associated in certain examples with a computer-assisted medical system used to perform a medical procedure on a body. To illustrate, FIG. 8 shows an illustrative computer-assisted medical system 800 that may be used to perform various types of medical procedures including surgical and/or non-surgical procedures.

As shown, computer-assisted medical system 800 may include a manipulator assembly 802 (a manipulator cart is shown in FIG. 8), a user control apparatus 804, and an auxiliary apparatus 806, all of which are communicatively coupled to each other. Computer-assisted medical system 800 may be utilized by a medical team to perform a computer-assisted medical procedure or other similar operation on a body of a patient 808 or on any other body as may serve a particular implementation. As shown, the medical team may include a first user 810-1 (such as a surgeon for a surgical procedure), a second user 810-2 (such as a patient-side assistant), a third user 810-3 (such as another assistant, a nurse, a trainee, etc.), and a fourth user 810-4 (such as an anesthesiologist for a surgical procedure), all of whom may be collectively referred to as users 810, and each of whom may control, interact with, or otherwise be a user of computer-assisted medical system 800. More, fewer, or alternative users may be present during a medical procedure as may serve a particular implementation. For example, team composition for different medical procedures, or for non-medical procedures, may differ and include users with different roles.

While FIG. 8 illustrates an ongoing minimally invasive medical procedure such as a minimally invasive surgical procedure, it will be understood that computer-assisted medical system 800 may similarly be used to perform open medical procedures or other types of operations. For example, operations such as exploratory imaging operations, mock medical procedures used for training purposes, and/or other operations may also be performed.

As shown in FIG. 8, manipulator assembly 802 may include one or more manipulator arms 812 (e.g., manipulator arms 812-1 through 812-4) to which one or more instruments may be coupled. The instruments may be used for a computer-assisted medical procedure on patient 808 (e.g., in a surgical example, by being at least partially inserted into patient 808 and manipulated within patient 808). While manipulator assembly 802 is depicted and described herein as including four manipulator arms 812, it will be recognized that manipulator assembly 802 may include a single manipulator arm 812 or any other number of manipulator arms as may serve a particular implementation. While the example of FIG. 8 illustrates manipulator arms 812 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 812 shown in FIG. 8.

During the medical operation, user control apparatus 804 may be configured to facilitate teleoperational control by user 810-1 of manipulator arms 812 and instruments attached to manipulator arms 812. To this end, user control apparatus 804 may provide user 810-1 with imagery of an operational area associated with patient 808 as captured by an imaging device. To facilitate control of instruments, user control apparatus 804 may include a set of master controls. These master controls may be manipulated by user 810-1 to control movement of the manipulator arms 812 or any instruments coupled to manipulator arms 812.

Auxiliary apparatus 806 may include one or more computing devices configured to perform auxiliary functions in support of the medical procedure, such as providing insufflation, electrocautery energy, illumination or other energy for imaging devices, image processing, or coordinating components of computer-assisted medical system 800. In some examples, auxiliary apparatus 806 may be configured with a display monitor 814 configured to display one or more user interfaces, or graphical or textual information in support of the medical procedure. In some instances, display monitor 814 may be implemented by a touchscreen display and provide user input functionality. Augmented content provided by a region-based augmentation system may be similar, or differ from, content associated with display monitor 814 or one or more display devices in the operation area (not shown).

Manipulator assembly 802, user control apparatus 804, and auxiliary apparatus 806 may be communicatively coupled to another in any suitable manner. For example, as shown in FIG. 8, manipulator assembly 802, user control apparatus 804, and auxiliary apparatus 806 may be communicatively coupled by way of control lines 816, which may represent any wired or wireless communication link as may serve a particular implementation. To this end, manipulator assembly 802, user control apparatus 804, and auxiliary apparatus 806 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory.

Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

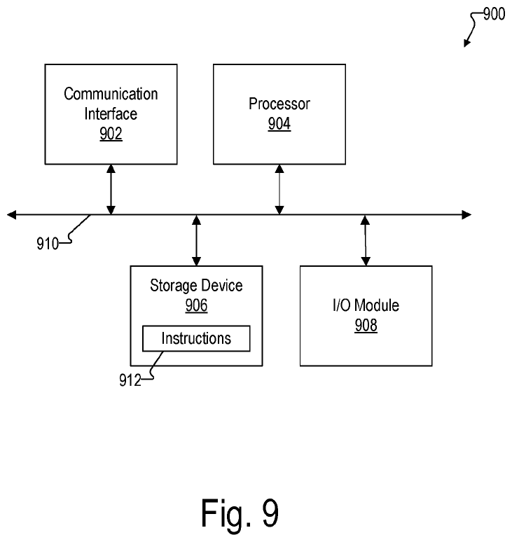

FIG. 9 shows an illustrative computing device 900 that may be specifically configured to perform one or more of the processes described herein. Any of the systems, computing devices, and/or other components described herein may be implemented by computing device 900.

As shown in FIG. 9, computing device 900 may include a communication interface 902, a processor 904, a storage device 906, and an input/output (“I/O”) module 908 communicatively connected one to another via a communication infrastructure 910. While an illustrative computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data and/or interpreting, executing, and/or directing execution of one or more of the instructions, processes, and/or operations described herein. Processor 904 may perform operations by executing computer-executable instructions 912 (e.g., an application, software, code, and/or other executable data instance) stored in storage device 906.

Storage device 906 may include one or more data storage media, devices, or configurations and may employ any type, form, and combination of data storage media and/or device. For example, storage device 906 may include, but is not limited to, any combination of the non-volatile media and/or volatile media described herein. Electronic data, including data described herein, may be temporarily and/or permanently stored in storage device 906. For example, data representative of computer-executable instructions 912 configured to direct processor 904 to perform any of the operations described herein may be stored within storage device 906. In some examples, data may be arranged in one or more databases residing within storage device 906.

I/O module 908 may include one or more I/O modules configured to receive user input and provide user output. I/O module 908 may include any hardware, firmware, software, or combination thereof supportive of input and output capabilities. For example, I/O module 908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
one or more processors communicatively coupled to the memory and configured to execute the instructions to perform a process comprising:
detecting an amount of deformation of a deformable three-dimensional (3D) model of a scene that occurs when an anatomical object located in the scene is deformed; and
determining, based on the amount of deformation of the deformable 3D model, a force value representative of a relative force associated with a change in the deformation of the anatomical object.

2. The system of claim 1, further comprising generating, based on imagery of the scene, the deformable 3D model.

3. The system of claim 2, wherein the generating the deformable 3D model includes using a simultaneous localization and mapping heuristic to generate the deformable 3D model as an imaging device captures imagery of the scene.

4. The system of claim 1, wherein the detecting the amount of deformation includes:
tracking, based on imagery of the scene, movement of a plurality of nodes representative of surface points on the anatomical object over time while the force is applied to the anatomical object;
updating 3D locations of vertices of the deformable 3D model that are associated with locations of the plurality of nodes; and
comparing the updated 3D locations of the vertices with previous 3D locations of the vertices.

5. The system of claim 4, wherein the imagery comprises stereoscopic images of the scene that are captured by an imaging device, and wherein the tracking movement of the plurality of nodes includes determining a depth of the plurality of nodes by processing the stereoscopic images.

6. (canceled)

7. (canceled)

8. The system of claim 1, wherein the determining the force value includes using a machine learning algorithm to identify a type of the anatomical object.

9. The system of claim 1, wherein the determining the force value is further based on one or more material property values for the deformable 3D model, the one or more material property values representative of one or more material properties of the anatomical object.

10. The system of claim 9, wherein the one or more material property values include:
a first material property value representative of a first material property associated with a first region of the anatomical object; and
a second material property value representative of a second material property associated with a second region of the anatomical object.

11. The system of claim 10, wherein the detecting the amount of deformation includes:
detecting a first amount of deformation of the deformable 3D model associated with the first region; and
detecting a second amount of deformation of the deformable 3D model associated with the second region.

12. The system of claim 9, wherein the one or more material property values include:
a first material property value representative of a material property associated with a first region of the anatomical object; and
a second material property value representative of the same material property associated with a second region of the anatomical object.

13. The system of claim 1, wherein the determining the force value is further based on a position of a physical tool applying the force to cause the deformation of the anatomical object.

14. The system of claim 13, further comprising detecting the position of the physical tool based on kinematic data representative of movement of the physical tool over time, the kinematic data generated by a computer-assisted medical system communicatively coupled with the physical tool.

15. The system of claim 13, wherein the determining the force value is further based on detecting the amount of deformation due to the position of the physical tool.

16. The system of claim 1, wherein the process further comprises determining a movement of a physical tool applying the force to cause the deformation of the anatomical object, wherein the determining the force value is further based on the movement of the physical tool.

17. The system of claim 16, wherein the process further comprises determining an amount of tool-induced deformation based on the amount of deformation of the deformable 3D model and the movement of the physical tool, wherein the determining the force value is based on the amount of tool-induced deformation.

18. The system of claim 16, wherein the determining the movement of the physical tool is based on kinematic data representative of movement of the physical tool over time, the kinematic data generated by a computer-assisted medical system communicatively coupled with the physical tool.

19-25. (canceled)

26. A method comprising:
detecting, by at least one computing device, an amount of deformation of a deformable three-dimensional (3D) model of a scene that occurs when an anatomical object located in the scene is deformed; and
determining, by the at least one computing device and based on the amount of deformation of the deformable 3D model, a force value representative of a relative force associated with a change in the deformation of the anatomical object.

27. The method of claim 26, further comprising generating, based on imagery of the scene, the deformable 3D model.

28. The method of claim 27, wherein the generating the deformable 3D model includes using a simultaneous localization and mapping heuristic to generate the deformable 3D model as an imaging device captures imagery of the scene.

29-50. (canceled)

51. A non-transitory computer-readable medium storing instructions that, when executed, direct a processor of a computing device to perform a process comprising:
detecting an amount of deformation of a deformable three-dimensional (3D) model of a scene that occurs when an anatomical object located in the scene is deformed; and
determining, based on the amount of deformation of the deformable 3D model, a force value representative of a relative force associated with a change in the deformation of the anatomical object.
